(19) United States
(12) Patent Application Publication
(10) Pub. No.: US 2024/0307416 A1
(43) Pub. Date: Sep. 19, 2024

(54) FILM-FORMING COMPOSITIONS COMPRISING SALICYLIC ACID AND METHODS OF USE

(71) Applicant: 3M INNOVATIVE PROPERTIES COMPANY, St. Paul, MN (US)

(72) Inventors: Katie F. Wlaschin, St. Paul, MN (US); Minghua Dai, St. Paul, MN (US)

(21) Appl. No.: 18/576,822

(22) PCT Filed: Jun. 29, 2022
(86) PCT No.: PCT/IB2022/056063

§ 371 Date: Jan. 5, 2024

Related U.S. Application Data
(60) Provisional application No. 63/226,978, filed on Jul. 29, 2021.

(51) Int. Cl. A61K 31/60; A61K 9/06; A61K 47/14; A61K 47/34

(52) U.S. Cl. CPC A61K 31/60; A61K 9/06; A61K 47/14; A61K 47/34

(57) ABSTRACT

Described herein is composition comprising salicylic acid for use as a treatment article for certain conditions and method of making such an article. The composition comprising: salicylic acid; a silicone containing film-forming polymer; a silicate tackifying resin; and an additive, wherein the additive comprises (i) a nonionic surfactant having an HLB of 5-9, (ii) a aminosilicone having an amine value greater than 60, (iii) a cationic silicone polyquaternium, or (iv) combinations thereof. Such compositions can be used for skin treatment applications.

### TECHNICAL FIELD

Film-forming compositions comprising salicylic acid are discussed along with methods of making the compositions and their use as a conformable skin treatment.

### SUMMARY

There is a desire to identify aesthetically pleasing conformable skin treatment products comprising salicylic acid.

In one aspect, a composition for use as a treatment article is described. The composition comprising:
salicylic acid;
a silicone containing film-forming polymer;
a silicate tackifying resin; and
an additive, wherein the additive is (i) a nonionic surfactant having an HLB of 5-9, (ii) an aminosilicone having an amine ratio greater than 0.05, (iii) a polyquaternium, or (iv) combinations thereof.

In one embodiment, the composition is used to treat acne. In another embodiment, the composition is used to treat warts.

In another aspect, method of making a gel composition is described. The method comprising:
combining a first part and a second part to make the gel composition, wherein the gel composition comprises
salicylic acid;
a silicone containing film-forming polymer;
a silicate tackifying resin;
a volatile solvent; and
an additive, wherein the additive comprises (i) a nonionic surfactant having an HLB of 5-9, (ii) an aminosilicone having an amine ratio greater than 0.05, (iii) a silicone-containing polyquaternium, or (iv) combinations thereof;
   wherein the first part comprises the salicylic acid in a first portion of the silicone containing, film-forming polymer; and the second part comprising a second portion of the silicone containing, film-forming polymer.

The above summary is not intended to describe each embodiment. The details of one or more embodiments of the invention are also set forth in the description below. Other features, objects, and advantages will be apparent from the description and from the claims.

### DETAILED DESCRIPTION

As used herein, the term
“a”, “an”, and “the” are used interchangeably and mean one or more; and “and/or” is used to indicate one or both stated cases may occur, for example A and/or B includes, (A and B) and (A or B);
“backbone” refers to the main continuous chain of the polymer;
“crosslinked” refers to connecting two pre-formed polymer chains using chemical bonds or chemical groups;
“interpolymerized” refers to monomers that are polymerized together to form a polymer backbone;
“monomer” is a molecule which can undergo polymerization which then form part of the essential structure of a polymer;
“polymer” refers to a macrostructure having a number average molecular weight (Mn) of at least 50,000 Dalton, at least 100,000 Dalton, at least 300,000 Dalton, at least 500,000 Dalton, at least, 750,000 Dalton, at least 1,000,000 Dalton, or even at least 1,500,000 Dalton as measured using techniques known in the art such as gel permeation chromatography; and not such a high molecular weight as to cause premature gelling of the polymer.

The term “polydiorganosiloxane” refers to a divalent segment of formula

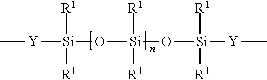

where each R¹ is independently an alkyl, haloalkyl, aralkyl, alkenyl, aryl, or aryl substituted with an alkyl, alkoxy, or halo; each Y is independently an alkylene, aralkylene, or a combination thereof; and subscript n is independently an integer of 0 to 1500.

As used herein, “film-forming” refers to a composition when allowed to dry under ambient conditions (e.g., 23° C. and 50% relative humidity (RH)) on skin or mucosal tissue forms a continuous layer that does not flake off after simple flexing of the tissue.

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

As used herein, “comprises at least one of” A, B, and C refers to element A by itself, element B by itself, element C by itself, A and B, A and C, B and C, and a combination of all three.

Silicone containing film-forming polymers are known to be used for making conformable bandages. The present disclosure is directed toward using a similar composition to make more aesthetically pleasing treatment options for skin treatments such as acne, warts, calluses, psoriasis, ringworm, and ichthyosis. However, as exemplified in U.S. Pat. No. 10,603,405 (Langer-Anderson et al.), all components except for the higher viscosity silicone containing film-forming polymer (e.g., SPOx) are combined together in the primary solvent (e.g., hexamethyldisiloxane) to generate a homogeneous mixture and then the SPOx is added last to increase the viscosity of the formula to the consistency of the finished gel. It has been discovered that when salicylic acid was added to the formulation of U.S. Pat. No. 10,603,405, wherein SPOx is added at the end of the manufacture and builds the viscosity, the salicylic acid did not appear dissolve and remained a solid particulate in the solution after manufacture. As it is important for the functional component (i.e., salicylic acid) to be uniformly dispersed in the gel, it was discovered that the order of mixing the components and the nature of the components (e.g., the additive) was critical to achieving a uniform gel solution wherein the composition was manufactured under a reasonable processing time. Ideally, the resulting composition (i.e., the gel and/or the dried film) is a single phase composition with no visible solids present, especially no solid salicylic acid particulates.

The composition of the present disclosure comprises: (a) salicylic acid; (b) a silicone containing film-forming polymer; (c) a silicate tackifying resin; and (d) an additive, wherein the additive is (i) a nonionic surfactant having an HLB of 5-9, (ii) a aminosilicone having an amine value greater than 60, (iii) a silicone polyquaternium, or (iv) combinations thereof. A volatile solvent is used to make a conformable gel composition, which upon evaporation, yields a treatment film.

### Salicylic Acid

The compositions of the present disclosure comprise salicylic acid. In one embodiment, the composition comprises at least 0.1, 0.5, or even 1% by weight of the salicylic acid and at most 3, 5, 7, or even 10% by weight. In one embodiment, the composition comprises at least 5, 8, 10, 15, or even 18% by weight of the salicylic acid and at most 20, 25, 30 or even 35% by weight. The amount of salicylic acid in the composition can vary based on whether the composition is in its gel or dried form and what skin condition the composition is directed toward.

### Film-Forming Polymer

Generally, the bulk of the composition comprises a film-forming polymer. The film-forming polymer is capable of forming a substantially continuous layer upon drying. Suitable film-forming polymers are at least partially soluble in a volatile solvent, and include silicone-containing polymers. Particularly suitable silicone containing polymers include polysiloxane polyamides. silicone polyureas, and silicone polyamines.

The film-forming polymer is typically soluble in the solvent system used in the gel composition. As used herein, a polymer is “soluble” or “solubilized” if the amount of polymer present in the solvent system is completely dissolved in the solvent system without the polymer forming a precipitate or visible, swollen gel particles in solution. As used herein, the term “solubility limit” is the maximum amount, measured as a percentage of the total weight of the solution, of a given polymer that can be dissolved in a given solvent system. For example, the film-forming polymer can have a solubility limit of at least 5, 10, 15, or even 20 wt % in the hexamethyldisiloxane, isooctane or any other solvent system described herein, based on the total weight of the gel composition.

Silicone-containing polymers useful for practicing the present disclosure may have an intrinsic viscosity (“IV”) of at least 0.9, 1.45, 1.68, or at least 1.8 as measured by the Inherent Viscosity Test Method of U.S. Pat. No. 8,765,881 (Hayes et al.). The silicone containing polymer typically has an intrinsic viscosity less than 3, as polymers having an intrinsic viscosity above 3 can be difficult to solubilize in certain circumstances. Lower IV polymers have notably higher solubility in the solvents and solvent systems and hence, while they can be film formers, they can be slower to dry and remain tacky after application. The IV of the polymers may be controlled during the polymerization of the polymer by varying initiator, initiator concentration, reaction temperature, reaction solvent, reaction method, and other parameters known to those skilled in the art.

In one embodiment, suitable silicone containing polymers include siloxanes and polysiloxane polyamides. Siloxane polymers have unique properties derived mainly from the physical and chemical characteristics of the siloxane bond. These properties include low glass transition temperature, thermal and oxidative stability, resistance to ultraviolet radiation, low surface energy and hydrophobicity. The siloxane polymers, however, often lack tensile strength. The low tensile strength of the siloxane polymers can be improved by forming block copolymers. Some block copolymers contain a “soft” siloxane polymeric block or segment and any of a variety of “hard” blocks or segments. Particularly suitable elastomeric siloxane-based elastomeric polymers are the segmented polymers of Formula I and Formula II below.

In some embodiments, the silicone-containing polymer is a linear polydiorganosiloxane, a linear polydiorganosiloxane polyamide block copolymer, or a polydiorganosiloxane urethane-containing copolymer, but other silicone-containing polymers may be useful.

A polydiorganosiloxane can have a variety of organic substituents on the silicon carbon atoms of the polysiloxane. For example, each organic substituent can be independently an alkyl, haloalkyl, arylalkylenyl, alkylarylenyl, alkenyl, aryl, or aryl substituted with an alkyl, alkoxy, or halo. The polydiorganosiloxane may have repeating units of the general formula (Si(R⁷)₂O—) wherein R⁷ is as defined below for any of the embodiments of R⁷ in Formula I. Examples include dimethylsilicones, diethylsilicones, and diphenylsilicones. In some embodiments, at least 40, 50, 60, 70, 80, 90, 95, 98, or even 99% of the R⁷ groups can be phenyl, methyl, or combinations thereof. In some embodiments, at least 40, 50, 60, 70, 80, 90, 95, 98, or even 99% of the R⁷ groups are methyl. High molecular weight polydimethylsiloxane (PDMS) for example having a molecular weight of at least 30,000 grams/mole is commercially available, for example, from Gelest Inc. Morrisville, PA.

A linear, polydiorganosiloxane polyamide block copolymer useful for practicing the present disclosure contains at least two repeat units of Formula I:

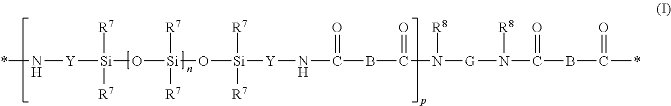

In this formula, each R⁷ is independently an alkyl, haloalkyl, arylalkylenyl, alkylarylenyl, alkenyl, aryl, or aryl substituted with an alkyl, alkoxy, or halo. Each Y is independently an alkylene, arylalkylene, alkylarylene, or a combination thereof. Subscript n is independently in a range from 0 to 1500 and subscript p is in a range from 1 to 10. Each group B is independently a covalent bond, an alkylene, an arylalkylene, an alkylarylene, an arylene, or a combination thereof. When each group B is a covalent bond, the polydiorganosiloxane polyamide block copolymer of Formula I is referred to as a polydiorganosiloxane polyoxamide block copolymer.

Group G is a divalent group that is the residue unit that is equal to a diamine of formula R⁸HN-G-NHR⁸ minus the two —NHR⁸ groups. Group R⁸ is hydrogen or alkyl (e.g., an alkyl having 1 to 10, 1 to 6, or 1 to 4 carbon atoms) or R⁸ taken together with G and with the nitrogen to which they are both attached forms a heterocyclic group. Each asterisk (*) indicates a site of attachment of the repeat unit to another group in the copolymer such as, for example, another repeat unit of Formula I.

Suitable alkyl groups for R⁷ in Formula I typically have 1 to 10, 1 to 6, or 1 to 4 carbon atoms. Examples of useful alkyl groups include methyl, ethyl, isopropyl, n-propyl, n-butyl, and iso-butyl. Suitable haloalkyl groups for R⁷ often have only a portion of the hydrogen atoms of the corresponding alkyl group replaced with a halogen. Examples of haloalkyl groups include chloroalkyl and fluoroalkyl groups with 1 to 3 halo atoms and 3 to 10 carbon atoms. Suitable alkenyl groups for R⁷ often have 2 to 10 carbon atoms. Examples of alkenyl groups often have 2 to 8, 2 to 6, or 2 to 4 carbon atoms such as ethenyl, n-propenyl, and n-butenyl. Suitable aryl groups for R⁷ often have 6 to 12 carbon atoms. Phenyl is an example of an aryl group. The aryl group can be unsubstituted or substituted with an alkyl (i.e., it may be an alklyarylenyl group) (the alkyl group may be, e.g., an alkyl having 1 to 10 carbon atoms, 1 to 6 carbon atoms, or 1 to 4 carbon atoms), an alkoxy (e.g., an alkoxy having 1 to 10 carbon atoms, 1 to 6 carbon atoms, or 1 to 4 carbon atoms), or halo (e.g., chloro, bromo, or fluoro). Suitable arylalkylenyl and alkylarylenyl groups for R⁷ usually have an alkylene group with 1 to 10 carbon atoms and an aryl group with 6 to 12 carbon atoms. In some arylalkylenyl and alkylarylenyl groups, the aryl group is phenyl and the alkylene group has 1 to 10 carbon atoms, 1 to 6 carbon atoms, or 1 to 4 carbon atoms. For example, R⁷ may be an arylalkylenyl group where any of these alkylene groups is bonded to a phenyl group.

In some embodiments, in some repeat units of Formula I, at least 40%, and in some embodiments at least 50%, of the R⁷ groups are phenyl, methyl, or combinations thereof. For example, at least 60, 70, 80, 90, 95, 98, or even 99% of the R⁷ groups can be phenyl, methyl, or combinations thereof. In some embodiments, in some repeat units of Formula I, at least 40 or even 50% of the R⁷ groups are methyl. For example, at least 60, 70, 80, 90, 95, 98, or even 99% of the R⁷ groups can be methyl. The remaining R⁷ groups can be selected from an alkyl having at least two carbon atoms, haloalkyl, arylalkylenyl, alkylarylenyl, alkenyl, aryl, or aryl substituted with an alkyl, alkoxy, or halo.

Each Y in Formula I is independently an alkylene, arylalkylene, alkylarylene, or a combination thereof. Suitable alkylene groups typically have up to 10, 8, 6, or even 4 carbon atoms. Examples of alkylene groups include methylene, ethylene, propylene, butylene, and the like. Suitable arylalkylene and alkylarylene groups usually have an arylene group with 6 to 12 carbon atoms bonded to an alkylene group with 1 to 10 carbon atoms. In some arylalkylene and alkylarylene groups, the arylene portion is phenylene. That is, the divalent arylalkylene or alkylarylene group has phenylene bonded to an alkylene having 1 to 10, 1 to 8, 1 to 6, or 1 to 4 carbon atoms. As used herein with reference to group Y, “a combination thereof” refers to a combination of two or more groups selected from an alkylene and arylalkylene or alkylarylene group. A combination can be, for example, a single alkylarylene bonded to a single alkylene (e.g., alkylene-arylene-alkylene). In one example of an alkylene-arylene-alkylene combination, the arylene is phenylene and each alkylene has 1 to 10, 1 to 6, or even 1 to 4 carbon atoms.

Each subscript n in Formula I is independently in a range from 0 to 1500. For example, subscript n can be up to 1000, 500, 400, 300, 200, 100, 80, 60, 40, 20, or even 10. The value of n is often at least 1, 2, 3, 5, 10, 20, or even 40. For example, subscript n can be in the range of 40 to 1500, 0 to 1000, 40 to 1000, 0 to 500, 1 to 500, 40 to 500, 1 to 400, 1 to 300, 1 to 200, 1 to 100, 1 to 80, 1 to 40, or even 1 to 20.

The subscript p is in a range from 1 to 10. For example, the value of p is often an integer up to 9, 8, 7, 6, 5, 4, 3, or even 2. The value of p can be in the range of 1 to 8, 1 to 6, or even 1 to 4.

Group G in Formula I is a residual unit that is equal to a diamine compound of formula R⁸HN-G-NHR⁸ minus the two amino groups (i.e., —NHR⁸ groups). The diamine can have primary or secondary amino groups. Group R⁸ is hydrogen or alkyl (e.g., an alkyl having 1 to 10, 1 to 6, or even 1 to 4 carbon atoms) or R⁸ taken together with G and with the nitrogen to which they are both attached forms a heterocyclic group (e.g., a 5- to 7-membered ring). In some embodiments, R⁸HN-G-NHR⁸ is piperazine. In some embodiments. R⁸ is hydrogen or an alkyl. In some embodiments, both of the amino groups of the diamine are primary amino groups (i.e., both R⁸ groups are hydrogen) and the diamine is represented by formula H₂N-G-NH₂.

In some embodiments, G is an alkylene, heteroalkylene, polydiorganosiloxane, arylene, arylalkylene, alkylarylene, or a combination thereof. Suitable alkylenes often have 2 to 10, 2 to 6, or even 2 to 4 carbon atoms. Examples of alkylene groups include ethylene, propylene, and butylene. Suitable heteroalkylenes are often polyoxyalkylenes such as polyoxyethylene having at least 2 ethylene units, polyoxypropylene having at least 2 propylene units, or copolymers thereof. Examples of polydiorganosiloxanes include polydimethylsiloxanes with alkylene terminal groups.

Suitable arylalkylene groups usually contain an arylene group having 6 to 12 carbon atoms bonded to an alkylene group having 1 to 10 carbon atoms. Some examples of arylalkylene groups are phenylene-alkylene where the phenylene is bonded to an alkylene having 1 to 10 carbon atoms, 1 to 8 carbon atoms, 1 to 6 carbon atoms, or even 1 to 4 carbon atoms. Some examples of alkylarylene groups are alkylene-phenylene, where the alkylene having 1 to 10 carbon atoms, 1 to 8 carbon atoms, 1 to 6 carbon atoms, or even 1 to 4 carbon atoms, is bonded to a phenylene. As used herein with reference to group G, “a combination thereof” refers to a combination of two or more groups selected from an alkylene, heteroalkylene, polydiorganosiloxane, arylene, arylalkylene, and alkylarylene. A combination can be, for example, an arylalkylene bonded to an alkylene (e.g., alkylene-arylene-alkylene). In one example of an alkylene-arylene-alkylene combination, the arylene is phenylene and each alkylene has 1 to 10, 1 to 6, or even 1 to 4 carbon atoms.

In some embodiments, the polydiorganosiloxane polyamide is a polydiorganosiloxane polyoxamide. The polydiorganosiloxane polyoxamide tends to be free of groups having a formula -B-(CO)—NH— where B is an alkylene.

All of the carbonylamino groups along the backbone of the copolymeric material typically are part of an oxalylamino group (i.e., the —(CO)—(CO)—NH— group), and B is a bond. That is, any carbonyl group along the backbone of the copolymeric material is bonded to another carbonyl group and is part of an oxalyl group. More specifically, the polydiorganosiloxane polyoxamide has a plurality of aminoxalylamino groups.

The polydiorganosiloxane polyamide is a block copolymer and can be an elastomeric material. Unlike many of the known polydiorganosiloxane polyamides that are generally formulated as brittle solids or hard plastics, the polydiorganosiloxane polyamides can be formulated to include greater than 50 weight percent polydiorganosiloxane segments based on the weight of the copolymer. The weight percent of the diorganosiloxane in the polydiorganosiloxane polyamides can be increased by using higher molecular weight polydiorganosiloxanes segments to provide greater than 60, 70, 80, 90, 95, or even 98% by weight of the polydiorganosiloxane segments in the polydiorganosiloxane polyamides. Higher amounts of the polydiorganosiloxane can be used to prepare elastomeric materials with lower modulus while maintaining reasonable strength.

Some of the polydiorganosiloxane polyamides can be heated to a temperature up to 200, 225, 250, 275, or even 300°° C. without noticeable degradation of the material. For example, when heated in a thermogravimetric analyzer in the presence of air, the copolymers often have less than a 10 percent weight loss when scanned at a rate 50° C. per minute in the range of 20° C. to 350° C. Additionally, the copolymers can often be heated at a temperature such as 250° C. for 1 hour in air without apparent degradation as determined by no detectable loss of mechanical strength upon cooling. The linear block copolymers having repeat units of Formula I can be prepared, for example by reaction of at least one polydiorganosiloxane-containing precursor with at least one diamine as described in U.S. Pat. No. 7,371,464; incorporated herein by reference.

The diamines are sometimes classified as organic diamines or polydiorganosiloxane diamines with the organic diamines including, for example, those selected from alkylene diamines, heteroalkylene diamines (such as polyoxyalkylene diamines), arylene diamines, aralkylene diamines, or alkylene-aralkylene diamines. The diamine has only two amino groups so that the resulting polydiorganosiloxane polyoxamides are linear block copolymers that are often elastomeric, hot melt processable (e.g., the copolymers can be processed at elevated temperatures such as up to 250° C. or higher without apparent degradation of the composition), and soluble in some common organic solvents. In some embodiments, the diamine is free of a polyamine having more than two primary or secondary amino groups. Tertiary amines that do not react with the polydiorganosiloxane-containing precursor of can also be present. Additionally, the diamines utilized in the reaction are free of any carbonylamino group. That is, the diamine is not an amide.

Preferred alkylene diamines (i.e., G is a alkylene) include, but are not limited to, ethylene diamine, propylene diamine, butylene diamine, hexamethylene diamine, 2-methylpentamethylene 1,5-diamine (i.e., commercially available from DuPont, Wilmington, DE under the trade designation DYTEK A), 1,3-pentane diamine (commercially available from DuPont under the trade designation DYTEK EP), 1,4-cyclohexane diamine, 1,2-cyclohexane diamine (commercially available from DuPont under the trade designation DHC-99), 4,4′-bis(aminocyclohexyl)methane, and 3-aminomethyl-3,5,5-trimethylcyclohexylamine.

The polydiorganosiloxane polyoxamide copolymer can be produced using a plurality of polydiorganosiloxane precursors, a plurality of diamines, or a combination thereof. A plurality of precursors having different average molecular weights can be combined under reaction conditions with a single diamine or with multiple diamines. For example, the precursor of may include a mixture of materials with different values of n, different values of p, or different values of both n and p. The multiple diamines can include, for example, a first diamine that is an organic diamine and a second diamine that is a polydiorganosiloxane diamine. Likewise, a single precursor can be combined under reaction conditions with multiple diamines.

The polydiorganosiloxane-containing precursor can be prepared by any known method. In some embodiments, this precursor is prepared according to the following reaction scheme, as described in previously cited U.S. Pat. No. 7,371,464 (Sherman et al.).

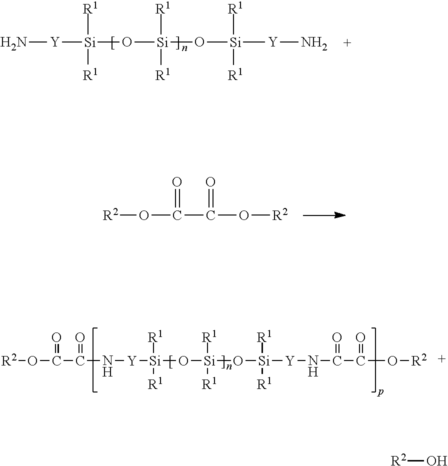

The polydiorganosiloxane diamine can be prepared by any known method and can have any suitable molecular weight.

Further details on suitable polydiorganosiloxane polyamides (including polydiorganosiloxane diamines and particularly polydiorganosiloxane polyoxamide) may be found, for example, among U.S. Pat. No. 8,586,668 (Leir et al), U.S. Pat. No. 5,214,119 (Leir et al.), U.S. Pat. No. 5,461,134 (Leir et al.), U.S. Pat. No. 5,512,650 (Leir et al.), and U.S. Pat. No. 7,371,464 (Sherman et al.), as well as U.S. Pat. Nos. 7,705,101 and 8,431,671 (Sherman et al.). Some polydiorganosiloxane diamines are commercially available, for example, from Shin Etsu Silicones of America, Inc., Torrance, CA and from Gelest Inc., Morrisville, PA.

Other examples of suitable silicone elastomers include polydiorganosiloxane polyurea copolymers and blends thereof, such as those described in U.S. Pat. Nos. 5,461,134 and 6,007,914 (Joseph et al.). Silicone-polyurethane copolymers (SPU) useful as film-forming polymers in the compositions and methods according to the present disclosure include block copolymers comprising silicone blocks and second blocks derived from a multifunctional isocyanate. At points herein the term silicone-polyurea may be used interchangeable with silicone-polyurethane. Useful silicone polyurea block copolymers are disclosed in, e.g., U.S. Pat. Nos. 5,512,650, 5,214,119, and 5,461,134, and 6,569,521, 6,664,359 (Melancon et al.) as well as International Publication Nos. WO 96/35458, WO 98/17726, WO 96/34028, WO 96/34030 and WO 97/40103.

Silicone blocks include those having the general formula (Si(R⁷)₂O—) wherein R⁷ is as defined above for any of the embodiments of R⁷ in Formula I. Non-limiting examples include dimethylsilicones, diethylsilicones, and diphenylsilicones.

Polydiorganosiloxane urethane-containing copolymers (a subset of the class of SPU materials) useful in compositions of the present disclosure contain soft polydiorganosiloxane units, hard polyisocyanate residue units, terminal groups and optionally soft and/or hard organic polyamine residue units. Some polydiorganosiloxane urea-containing copolymers are commercially available under the trade designation “GENIOMER 140” available from Wacker Chemie AG, Germany. The polyisocyanate residue is the polyisocyanate minus the —NCO groups, the organic polyamine residue is the organic polyamine minus the-NH groups, and the polyisocyanate residue is connected to the polydiorganosiloxane units or organic polyamine residues by urea linkages. The terminal groups may be non-functional groups or functional groups depending on the purpose of the polydiorganosiloxane urea segmented copolymer.

In some embodiments, the polydiorganosiloxane urethane containing copolymers useful as polymer processing additives contain at least two repeat units of Formula II

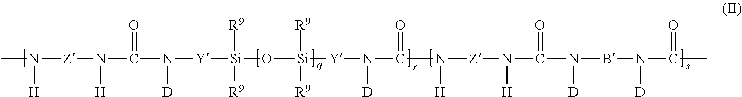

In this Formula II each R⁹ is a moiety that independently is an alkyl, cycloalkyl, aryl, perfluoroalkyl, or a perfluoroether group. In some embodiments of R⁹, alkyl has about 1 to 12 carbon atoms and may be substituted with, for example, trifluoroalkyl, vinyl, a vinyl radical or higher alkenyl represented by the formula —R¹⁰(CH₂)ₐCH═CH₂ wherein R¹⁰ is —(CH₂)_{b}— or —(CH₂)_{c}CH—CH— and a is 1, 2 or 3; b is 0, 3 or 6; and c is 3, 4 or 5. In some embodiments of R⁹. cycloalkyl has about 6 to 12 carbon atoms and may be substituted with one or more alkyl, fluoroalkyl, or vinyl groups. In some embodiments of R⁹, aryl has about 6 to 20 carbon atoms and may be substituted with, for example, alkyl, cycloalkyl, fluoroalkyl and vinyl groups. In some embodiments of R⁹, the perfluoroalkyl group is as described in U.S. Pat. No. 5,028,679, wherein such description is incorporated herein by reference, and the perfluoroether-containing group is as described in U.S. Pat. Nos. 4,900,474 and 5,118,775, wherein such descriptions are incorporated herein by reference. In some embodiments, R⁹ is a fluorine-containing group is as described in U.S. Pat. No. 5,236,997, wherein such description is incorporated herein by reference. In some embodiments, at least 50% of the R⁹ moicties are methyl radicals with the balance being monovalent alkyl or substituted alkyl radicals having 1 to 12 carbon atoms, alkenylene radicals, phenyl radicals, or substituted phenyl radicals. In Formula II, each Z′ is arylene, arylalkylene, alkylene, or cycloalkylene. In some embodiments of Z′, the arylene or arylalkylene has from about 6 to 20 carbon atoms. In some embodiments of Z′, alkylene or cycloalkylene radical has from about 6 to 20 carbon atoms. In some embodiments, Z′ is 2,6-tolylene, 4,4′-methylenediphenylene, 3,3′-dimethoxy-4,4′-biphenylene, tetramethyl-m-xylylene, 4,4′-methylenedicyclohexylene, 3,5,5-trimethyl-3-methylenecyclohexylene, 1,6-hexamethylene, 1,4-cyclohexylene, 2,2,4-trimethylhexylene, or mixtures thereof. In Formula II, each Y′ is independently alkylene, arylalkylene, alkylarylene, or arylene. In some embodiments of Y″, alkylene has from 1 to 10 carbon atoms. In some embodiments of Y′, the arylalkylene, alkylarylene, or arylene has from 6 to 20 carbon atoms. In Formula II, each D is independently hydrogen, an alkyl radical having 1 to 10 carbon atoms, phenyl, or a radical that completes a ring structure including B′ or Y′ to form a heterocycle. In Formula II, B is a polyvalent radical selected from the group consisting of alkylene, arylalkylene, alkylarylene, cycloalkylene, phenylene, polyalkylene oxide (e.g., polyethylene oxide, polypropylene oxide, polytetramethylene oxide, and copolymers and mixtures thereof). In Formula II, “s” is a number that is 0 to about 1000; “r” is a number that is equal to or greater than 1; and “q” is a number that is about 5 or larger, in some embodiments about 15 to 2000, and in some embodiments about 30 to 1500.

In the use of polyisocyanates (Z′ is a radical having a functionality greater than 2) and polyamines (B′ is a radical having a functionality greater than 2), the structure of Formula II will be modified to reflect branching at the polymer backbone. In the use of endcapping agents, the structure of Formula II will be modified to reflect termination of the polydiorganosiloxane urea chain.

The linear block copolymers having repeat units of Formula I and polymdiorganolsiloxane urea containing polymers of Formula II can be prepared, for example, as discussed in U.S. Pat. No. 8,552,136 (Papp et al.).

Other examples of silicone containing polymers include those formed from silanols, silicone hydrides, siloxanes, epoxides, and (meth)acrylates. When the film-forming polymer is prepared from (meth)acrylate-functional siloxanes, the polymer is sometimes referred to as a siloxane (meth)acrylate. Additionally, other amphiphilic siloxy-containing polymers have been reported as useful in gel compositions (U.S. Pat. No. 7,795,326 (Salamone et al.)), wherein the hydrophobic siloxysilane monomer is copolymerized with a hydrophilic nitrogen-containing monomer. Other siloxy-containing polymers include block copolymers of polydimethylsiloxane and polyurethane, and block copolymers of polydimethylsiloxane and poly(ethylene glycol). Still, other potentially viable film-forming polymers include block copolymers of polystyrene and ethylene/butylene, block copolymers of polystyrene and polyisobutylene, block copolymers of polystyrene and polyisoprene, block copolymers of polystyrene and polybutadiene, block copolymers of polydimethylsiloxane and polyurethanes, polymers of C4-C18 acrylates and methacrylates, butyl rubber, polyisobutylene, and combinations thereof.

Another suitable siloxy-containing monomer for certain gel compositions is based upon the siloxy monomer, 3-methacryloyloxypropyltris(trimethylsiloxy)silane (TRIS). TRIS can be used in combination with both hydrophilic comonomers, such as N-isopropylacrylamide (NIPAM), or hydrophobic comonomers, such as methyl methacrylate, such that the resulting copolymers are soluble in a volatile solvent.

The film-forming polymer is typically present in quantities of at least 5 wt. % and no

greater than 30 wt. %, based on the total weight of the gel composition, or any amount within that range. In certain implementations, it may be preferred that the film-forming polymer is present at a concentration of at least 5, 8, 10, or even 12 wt %; and at most 15, 20, 25, or even 30 wt % based on the total weight of the gel composition.

In one embodiment, a dried film cast form the gel composition may include an amount of film-forming polymer of at least 30, 35, 40, 45, or even 50 wt %; and at most 55, 60, 65, 70, 75, 80, 85, or even 90 wt % relative to a total weight of the dried film.

### Silicate Tackifying Resin

Silicate tackifying resins can be added to the film-forming polymer to provide or enhance the adhesive properties of the composition. The silicate tackifying resin can influence the physical properties of the resulting gel composition. For example, as silicate tackifying resin content is increased, the glassy to rubbery transition of the gel composition occurs at increasingly higher temperatures. In some exemplary gel compositions, a plurality of silicate tackifying resins can be used to achieve desired performance. Suitable silicate tackifying resins include those resins composed of the following structural units M (i.e., monovalent R′₃SiO_{1/2} units), D (i.e., divalent R′₂SiO_{2/2} units), T (i.e., trivalent R′SiO_{3/2} units), and Q (i.e., quaternary SiO_{4/2} units), and combinations thereof. Typical exemplary silicate resins include MQ silicate tackifying resins, MQD silicate tackifying resins, and MQT silicate tackifying resins. These silicate tackifying resins usually have a number average molecular weight in the range of 100 to 50,000 or in the range of 500 to 15,000 and generally have methyl R′ groups.

Such resins are described in, for example, Encyclopedia of Polymer Science and Engineering, vol. 15, John Wiley & Sons, New York, (1989), pp. 265-270, and U.S. Pat. No. 2,676,182 (Daudt et al.), U.S. Pat. No. 3,627,851 (Brady), U.S. Pat. No. 3,772,247 (Flannigan), and U.S. Pat. No. 5,248,739 (Schmidt et al.). Other examples are disclosed in U.S. Pat. No. 5,082,706 (Tangney). The above-described resins are generally prepared in solvent. Dried or solventless, M silicone tackifying resins can be prepared, as described in U.S. Pat. No. 5,319,040 (Wengrovius et al.), U.S. Pat. No. 5,302,685 (Tsumura et al.), and U.S. Pat. No. 4,935,484 (Wolfgruber et al.).

MQ silicate tackifying resins are particularly suitable for several gel compositions of the present disclosure. MQ silicate tackifying resins are copolymeric resins having R′₃SiO_{1/2} units (“M” units) and SiO_{4/2} units (“Q” units), where the M units are bonded to the Q units, each of which is bonded to at least one other Q unit. Some of the SiO_{4/2} units (“Q” units) are bonded to hydroxyl radicals resulting in HOSiO_{3/2} units (“TOH” units), thereby accounting for the silicon-bonded hydroxyl content of the silicate tackifying resin, and some are bonded only to other SiO_{4/2} units.

Certain MQ silicate tackifying resins can be prepared by the silica hydrosol capping process described in U.S. Pat. No. 2,676,182 (Daudt et al.) as modified according to U.S. Pat. No. 3,627,851 (Brady), and U.S. Pat. No. 3,772,247 (Flannigan).

In one embodiment, the silicate tackifying resin is added to the composition to at least 1, 2, 3, 4, 5, 8, 10, or even 15 wt %; and at most 35, 30, 25, 20, or even 15 wt % based on the total weight of the gel composition. In one embodiment, the silicate tackifying resin is added to the composition to at least 5, 8, 10, 15, or even 20 wt %; and at most 40, 35, 30, 25, or even 20 wt % based on a total weight of the dried film.

### Additive

The present disclosure comprises an additive, which is used, along with the silicone containing film-forming polymer, to incorporate salicylic acid uniformly into the gel composition, and resulting dried film. Most useful additives also either improve adhesion or do not result in reduced adhesion. The additive comprises a nonionic surfactant, an aminosilicone, a silicone polyquaternium, or combinations thereof.

In one embodiment, the additive is a nonionic surfactant having a hydrophilic-lipophilic balance (HLB) of at least 5, 6, or even 7; and at most 7.5, 8, 8.5, or even 9.

Any nonionic surfactant meeting the defined HLB range may be used so long as it is suitable for topical (e.g., skin) applications. Nonionic surfactants that may be particularly useful include: polyglyceryl esters having the defined HLB such as polyglyceryl-4-isostearate (available under the trade designation “ISOLAN GI 34” from Evonik, Essen, Germany), methylglucose isostearate (available under the trade designation “ISOLAN IS” from Evonik), polyglyceryl-6-disterate, and polyglyceryl-4-stearate; polyoxyethylene oleyl ethers having the defined HLB such as those available under the trade designation “BRIJ O3-LQ” and “BRIJ O5-LQ” from Crodo, Plainsboro, NJ; fatty acid monoesters of glycerin and propylene glycol such as glycerol monolaurate, glycerol monocaprylate, glycerol monocaprate, C₈-C₁₂ alkyl monoethers of glycerin and propylene glycol having the defined HLB such as 2-ethylhexyl glycerin ether (available from

Schuelke Mayr, Norderstedt, Germany, under the trade designation “SENSIVA SC 50”); polyethylene glycol ethers of lauryl alcohol such as Laureth-3 (2-[2-[2-(dodecyloxy)ethoxy]ethoxy]-ethanol); and 1,2-alkanediols having a chain length in the range of 5 to 10 carbon atoms having the defined HLB, such as 1,2-octanediol. One exemplary suitable 1,2-octanediol composition includes 3-[(2-ethylhexyl)oxy]-1,2-propanediol, and is sold as SENSIVA SC-10 by Schülke&Mayr GmbH, Germany.

In one embodiment, the additive is an aminosilicone. As used herein “aminosilicone” means any amine functionalized silicone; i.e., a silicone containing at least one primary amine, secondary amine, or tertiary amine, group. Typically, these are silicones which have been chemically modified so that some of the pendant groups along the backbone have been replaced with various alkylamine groups (—R—NH₂). These amine groups can become positively charged in aqueous solutions because of their electron-donating tendencies, yielding an inorganic, cationic polymer. Useful aminosilicones are typically water soluble or water-dispersible.

In one embodiment, the aminosilicone having an amine value greater than 60, 80, 100, or even 150 appears to aid the solubilization of the salicylic acid. In one embodiment, the aminosilicone having an amine value no more than 60. In the present disclosure, “amine value” represents the number of milliliters of 0.1N HCl needed to neutralize 10 g of the amine-rich adhesion promoter. Amine value is preferably calculated according to the following equation:

$$1 / FGMW * times 100 , 000 * FGMW = functional group molecular weight of the amine groups$$

Without wishing to be bound by theory, the amine content of the aminosilicone appears, among other parameters and characteristics, to be directly correlated with improved dissolution of the salicylic acid. Accordingly, aminosilicones suitable for use in the gel compositions of the present disclosure advantageously include a greater number of available amine groups and an accompanying larger amine value. Ideally, the aminosilicone has a higher amine value for a given polymer chain length. In one embodiment, the aminosilicone has a ratio of amine number to viscosity of less than 4, 2, 1, 0.5, 0.2, or even 0.1.

Exemplary aminosilicones for use in embodiments of the present disclosure can be linear polymers, branched polymers, copolymers, and mixtures thereof. In some embodiments the copolymer is a block copolymer. In some embodiments, including those of presently preferred compositions, the aminosilicone has one or more amine groups pendant from the polymer backbone. Examples of such embodiments are illustrated by compounds of formula IV with pendant mono-amines and compounds of Formula VI with pendant di-amines, as shown herein below. In some embodiments the polymer has amine groups at one or more termini of the polymer. Examples of such embodiments are illustrated by compounds of Formula V, as shown herein below. Aminosilicones may further be selected from the group comprising, aminodimethicones, trimethylsilylamodimethicones, aaminoethylaminopropylsiloxane-dimethylsloxane copolymers, and mixtures thereof.

In some embodiments, an aminosilicone has the structure of Formula IV:

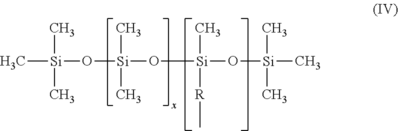

wherein R is C1-12 (preferably C1-6) alkyl, the blocks bearing the subscripts x and y may be randomly mixed, the total value of x is from 10 to 5,000, for example 58 or 100 or 118, and the total value of y is from 2 to 20, preferably 2 to 11, for example 4 or 11. In some embodiments x is 58 and y is 4; x is 100 and y is 4; or x is 118 and y is 11. In some embodiments, R is a linear C₃H₆ group.

In some embodiments, one or more aminosilicones have the structure of formula V, which features terminal amine groups:

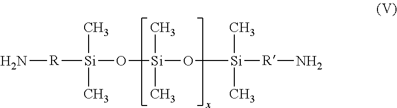

wherein x is from 5 to 5,000, and R and R′, which may be the same or different, are each saturated, linear or branched alkyl groups of 1 to 12 carbon atoms (in presently preferred circumstances, 1 to 6 carbon atoms), e.g., a linear C₃H₆ group.

In other embodiments the aminosilicone includes a branched diamino functional polydimethylsiloxane of formula VI:

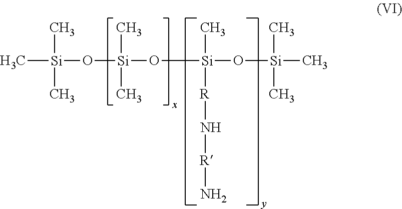

wherein the blocks bearing the subscripts x and y may be randomly mixed, the total value of is from 5 to 5,000, the total value of y is from 1 to 20, e.g., 8, and R and R′, which may be the same or different, are each saturated, linear or branched alkyl groups of 1 to 12 carbon atoms (preferably 1 to 6), e.g., R is a linear C₃H₆ group and R′is a linear C₂H₄ group.

In some embodiments, the aminosilicone is selected from the group comprising GP-4 (a compound of formula IV wherein R=(CH₂)₃, x=58 and y=4, available for example from Genesee Polymers Corporation (“GPC”), Burton, Michigan, USA, and having an amine value of about 90); GP-581 (a compound of formula IV wherein R=(CH₂)₃, x=118 and y=11, available for example from GPC and having an amine value of about 110); GP-965 (a compound of formula V wherein R=R′=(CH₂)₃, x=10, available for example from GPC and having an amine value of about 200); KF-393 (a diamino modified compound of formula IV having an amine value of about 286, available from Shin-Etsu Silicones); KF-8004 (a diamino modified compound of formula IV having an amine value of about 67, available from Shin-Etsu Silicones); a compound of formula VI wherein R=(CH₂)₃, R′=(CH₂)₂, having an amine value of about 230, available under the trade designation “SILAMINE AO EDA” from Siltech Corporation, Toronto, Ontario, Canada); a compound of formula VI wherein R=(CH₂)₃, R′=(CH₂)₂, having an amine value of about 170, available under the trade designation “SILAMINE D2 EDA” from Siltech Corporation); and amine value commercial alternatives thereof (such as amine silicones from other suppliers, as will be appreciated by persons skilled in the art), and mixtures thereof. In one embodiment, the additive is a cationic silicone polyquaternium. As used herein a “silicone polyquaternium” includes any silicone comprising one or more quaternary ammonium groups. Exemplary cationic silicone polyquaterniums include: silicone quaternium-12 (available, for example, under the trade designation “PECOSIL CA-1240”, a reaction product of cocamidopropyldimethylamine and Dimethicone PEG-7 acetyl chloride, from Phoenix Chemical, Somerville, NJ); silicone quaternium-8 (available, for example, under the trade designation “PECOSIL AD-3640” from Phoenix Chemical, Somerville, NJ); silicone quaternium-19 (available, for example, under the trade designation “ZENESTER Q”, a functionalized cationic polymeric silicone polyester made from the reaction of a cationic dimethicone copolyol and a dimer acid, from Zenitech, Toronto, Ontario); silicone quaternium-22 (available under the trade designation “ABIL T QUAT 60” from Evonik Industries AG, Essen, Germany); silicone quaternium-80 (available under the trade designation “ABIL T QUAT 3272” from Evonik Industries AG); and mixtures thereof.

In one embodiment, the additive is typically present in quantities of at least 0.05, 0.1, 0.5, 1, 1.25, 1.5, 1.75, 2, or even 2.25 wt % based on the total weight of the gel composition. In one embodiment, the additive is present in quantities of at most 2, 2.5, 3, 3.5, 4, 4.5, 5, 7, or even 10 wt % based on the total weight of the gel composition. The amount of additive present may be dictated by the amount of salicylic acid present, wherein the more salicylic acid present, the more additive is present.

A dried film cast from the gel composition may include, for example an amount of additive of at least 0.1, 0.5, 1, 1.5, 2, 2.25, 2.5, 3, 4, 5, 6, or even 8 wt % and at most 10, 15, 20, 25, or even 30 wt % relative to a total weight of the dried film.

### Solvent

The gel form of the composition comprises a volatile solvent. In one embodiment, the volatile solvent is selected from the group consisting of volatile linear and cyclic siloxanes, volatile polydimethylsiloxanes, isooctane, octane, and combinations thereof. The solvent is typically at least 40, 50, 55, or even 60 wt % and at most 65, 70, 75, or 80 wt % of the total gel composition.

As the composition may be applied to tissue, the solvent is desirably volatile and non-stinging. As used herein, “volatile” has its standard meaning, that is, it can evaporate rapidly at normal temperatures and pressure. For example, a solvent can be volatile if one metric drop ( 1/20 mL, 50 mu L) of the solvent will evaporate completely between 20-25° C. within 5 minutes, or within 4 minutes, or within 3 minutes, or within 2 minutes, or within 1 minute, or within 30 sec, or within 15 sec. Exemplary volatile solvent systems include a linear siloxane or a cyclic siloxane, such as hexamethyldisiloxane (HMDSO), octamethylcyclotetrasiloxane, decamethylcyclopentasiloxane, and octamethyltrisiloxane, or a linear, branched or cyclic alkane, such as propane, isobutane, liquid butane (e.g., under pressure), pentane, hexane, heptane, octane, petroleum distillates, cyclohexane, fluorocarbons, such as trichloromonofluoromethane, dichlorodifluoromethane, dichlorotetrafluoroethane, tetrafluoroethane, heptafluoropropane, 1,1-difluoroethane, pentafluoropropane, perfluoroheptane, perfluoromethylcyclohexane, 1,1,1,2,-tetrafluoroethane, 1,1,1,2,3,3,3-heptafluoropropane, chlorofluorocarbons, in addition to liquid carbon dioxide, and combinations thereof.

The use of a non-polar, volatile solvent, alone or in combination, as the primary liquid phase of the gel composition can provide a desirable balance between fast drying and reduced skin irritation during application. In presently preferred implementations, the solvent is one of HMDSO and isooctane. Other, more polar solvents such as ethanol, isopropanol, glycerin, N-methylpyrrolidone, and N,N-dimethylacetamide can be used in other implementations, where a non-stinging gel composition is either unnecessary or undesirable. Numerous aprotic solvents have utility including acetates such as methyl and ethyl acetate, propylene glycol diacetate, volatile ketones such as acetone and methyl ethyl ketone, volatile ethers such as diethyl ether, ethyl propyl ether, dipropyl ether and dipropylene glycol dimethyl ether, volatile fluorocarbons, such as pentafluoropropane, perfluoroheptane, perfluoromethylcyclohexane and the like; or a volatile gas, such as carbon dioxide, can also be employed, each with varying degrees of user discomfort.

In some implementations, water may be included in a solvent system. In certain implementations, a relatively small amount of water is present in the gel composition, such as at least 0.1 or even 1 wt. %, but no more than 5 or 10 wt % based on the total weight of the composition. Higher water content can be used in the gel compositions, though such compositions may require longer dry times.

### Optional Components

In addition to the components described above, optional components can be added to the composition to improve the performance of the composition. Optional components include: a coagulant, a filler, clay, silica, colorants and/or fibrous reinforcement as disclosed in U.S. Pat. No. 10,603,405, herein incorporated by reference.

In one embodiment, an antiseptic and/or antibiotic agent may be added to the composition. Such agents can be used for shelf-life preservation of the composition and/or to supplement the effectiveness of the salicylic acid for various treatments. Such agents are described in U.S. Pat. No. 10,603,405 Exemplary agents include, benzethonium chloride, cetylpyridinium chloride, benzalkonium chloride, chlorhexidine, polyhexamethylene biguanide, chloroxylenol, methylparaben, and propylparaben

Other solid biologically active materials, such as anti-itch agents, such as chamomile, eucalyptus, camphor, menthol, zinc oxide, talc, and calamine, anti-inflammatory agents, such as corticosteroids, antifungal agents, such as terbinafine hydrochloride and miconazole nitrate, and non-steriodal anti-inflammatory agents, such as ibuprofen, and antibiotic agents, such as bacitracin, neomycin, polymyxin can be added in like fashion. Essential oils can also be added as flavoring agents, aromatic agents, or antimicrobial agents, including thymol, menthol, sandalwood, cinnamon, jasmine, lavender, pine, lemon, rose, eucalyptus, clove, orange, mint, spearmint, peppermint, lemongrass, bergamot, citronella, cypress, nutmeg, spruce, tea tree, wintergreen, vanilla, and the like.

In one embodiment, a colorant, such as a dye, pigment, or pigment dye maybe added to the composition to improve its aesthetical appearance. For example, making the composition skin-tone in color.

### Method of Making

Typically, conformable gel compositions comprising a film-forming polymer are made by

adding all of the components except for the silicone containing film-forming polymer together to homogeneously mix then and then add the silicone containing film-forming polymer at the end of the manufacturing process, making a viscous solution. As mentioned, salicylic acid does not appear to be readily soluble in the silicate tackifying resin, and/or the volatile solvent. Therefore, it has been discovered that certain additives and the addition of at least a portion of the silicone containing film-forming polymer to the salicylic acid can be used to solubilize the salicylic acid, while not creating too viscous of a solution. As can be seen in the Example Section below, particular additives assist in solubilizing the salicylic acid. Generally, the resulting gel composition and/or the film comprises few (i.e., less than 20, 10, 5, 2, or even 1 wt % of the amount initially added) salicylic acid particulates and more preferably no salicylic acid particulates. If the gel composition comprises a few salicylic particulates, the composition can be optimized (for example, increasing the amount of the additive, increasing the silicone containing film-forming polymer, and/or decreasing the amount of salicylic acid), and/or gently heating the composition to aid dissolution of the salicylic acid. The other components of the composition (e.g., a silicate tackifying resin, the additive, and optional other components) can be added to this first mixture comprising the salicylic acid and at least a portion of the silicone containing film-forming polymer, or they can be added separately. Generally, the remainder of the silicone containing film-forming polymer is added at the end of the mixing of the components due to its viscous nature.

Generally, the salicylic acid and at least a portion of the silicone containing film-forming polymer, and optionally the additive are mixed at ambient conditions using techniques known in the art, such as overhead mixer. The mixture may be slightly heated (for example, at least 40, 60, or even 80° C. and below the boiling/flash point of the solvent or 200° C., whichever is lower) to more quickly solubilize the salicylic acid.

The composition of the present disclosure can be useful in topical applications for treatment of acne, warts, psoriasis, ringworm, ichthyosis, and/or calluses.

A treatment protocol may involve skin preparation prior to applying the gel compositions of the present disclosure. The target site is preferably dried, e.g., blotted dry, and then a lightly adherent polymeric film is formed over this site by applying the gel composition.

Sufficient amounts of the composition are employed to cover (i.e., coat) the entire target site with a layer of the gel composition. It is typically preferred that the resultant dried film have a thickness from about 4 mils (101.2 micrometers) to about 15 mils (351 micrometers). The resultant film typically covers just the portion to be treated (for example, just the acne, wart, etc.). If necessary, excess gel can be removed with a wipe or tissue paper before drying. The gel composition may be applied as a single dose or multiple doses (applications).

As used herein, “film-forming” refers to a composition when allowed to dry under ambient conditions (e.g., 23° C. and 50% relative humidity (RH)) on skin or mucosal tissue forms a continuous layer that does not flake off after simple flexing of the tissue.

Typical gel compositions can comprise (a) 0.1 to 10 wt % salicylic acid; (b) 5 to 30 wt. % silicone containing film-forming polymer; (c) 1 to 35 wt. % silicate tackifying resin; (d) 0.1 to 5 wt % additive; and (e) 50 to 80 wt. % volatile solvent, based on the total weight of the gel composition.

The gel compositions can be applied to tissue and the volatile solvent evaporates forming a continuous dried film. Dried films of the present disclosure may comprise (a) 0.1 to 20 wt % salicylic acid; (b) 30 to 90 wt. % silicone containing film-forming polymer; (c) 5 to 40 wt. % silicate tackifying resin; and (d) 0.1 to 25 wt % additive, based on the total weight of the dried film.

As used herein “ready to use” refers to the composition intended to be applied (e.g., to skin) without dilution. It should be understood that (unless otherwise specified) the listed amounts of all identified components are for “ready to use” gel compositions.

The gel compositions of the present disclosure typically have a viscosity of at least 20,000 Centipoise (cps) and no greater than 1,100,000 cps and can encompass all values therebetween when measured at 23° C. using a Brookfield LVT viscometer as described in U.S. Pat. No. 10,603,405.

In one embodiment, a film of dried gel composition can have a thickness of at least 25, 50, 75, or even 100 micrometers and typically no greater than 0.2, 0.25, 0.5, 1, and 1.3 mm. While the gel compositions of the present disclosure can be coated in such a manner as to form a film having a uniform or substantially uniform thickness, variations in, for example, the pressure applied or the applicator used can result in variable thickness throughout the film layer.

In one embodiment, the dried films of the present disclosure are lightly adherent to skin. In one embodiment, similar in adhesion as described in U.S. Pat. No. 10,603,405.

Advantageously, the dried films of the present disclosure are self-supporting after a single application of gel composition, meaning that a therapeutic level of salicylic acid can be applied in a single strata and without the application of additional layers of gel composition on an outer surface of a dried film. Moreover, a “self-supporting” film does not require an additional, flexible backing for continued wear (i.e., at least 8 hours of continuous existence on the skin or other target tissues).

In one embodiment, the compositions of the present disclosure are useful in the treatment of acne. Propionibacterium acnes, now known as Cutibacterium acnes, is a bacteria often associated with acne skin conditions. In one embodiment of the present disclosure, the dried films of the present disclosure are able to achieve at least a 1, 3, 5, or even 6 log reduction using the Antimicrobial Efficacy Test described herein.

The dried films of the present disclosure should be capable of releasing the active agent such as salicylic acid during use. The films should not significantly irritate the skin when deposited during application and in use after drying. The dried films are substantially painless and can be removed, if needed, substantially without pain. The treatment films can form when applied over surfaces wet with water, blood or body fluids, in short times at standard room temperature and reasonable variants thereof.

### EXAMPLES

Unless otherwise noted, all parts, percentages, ratios, etc. in the examples and the rest of the specification are by weight, and all reagents used in the examples were obtained, or are available, from general chemical suppliers such as, for example, Sigma-Aldrich Company, Saint Louis, Missouri, or may be synthesized by conventional methods.

TABLE 1
Film-Forming Gel Components

| Component | Description and Source |
|---|---|
| Salicylic Acid | Sigma Aldrich Company, St. Louis, MO |
| Silicone Polyoxamide | Poly(diorganosiloxane)-polyoxamide copolymer |
| (SPOx) | made from a diamine of 25,000 molecular |
| | weight as per “Preparatory Example 1” of |
| | U.S. Pat. No. 7,947,376. |
| MQ Resin | Silicate tackifying resin (BELSIL TMS 803); |
| | Wacker Chemical, Chicago, IL |
| HMDSO | Hexamethyldisiloxane; Wacker Chemical |

TABLE 2
Nonionic Surfactant Additives

| Additive | HLB Value | Source |
|---|---|---|
| SENSIVA SC-10 (composition of | 7.0-7.5^{a} | Schülke & Mayr GmbH, |
| 1,2-octanediol,3-[(2- | | Germany |
| ethylhexyl)oxy]-1,2-propanediol) | | |
| Glycerol monolaurate (CAS No. 27215-38-9) | 7.0^{a} | Lonza, South Plainfield, NJ |
| SENSIVA SC-50 (ethylhexylglycerin, | 7.5^{a} | Schülke & Mayr GmbH, |
| CAS No. 70455-33-9) | | Germany |
| Polyglyceryl-6-distearate | 8.0^{a} | Lonza, South Plainfield, NJ |
| (CAS No. 34424-97-0) | | |
| Polyglyceryl-4-stearate | 6.0^{a} | Nikkol Group, Tokyo, Japan |
| (CAS No. 37349-34-1) | | |
| BRIJ O3-LQ (polyoxyethylene (3) oleyl ether) | 6.6^{a} | Croda, Plainsboro, NJ |
| BRIJ O5-LQ (polyoxyethylene (5) oleyl ether) | 9.1^{a} | Croda, Plainsboro, NJ |
| SPAN 20 (sorbitan monolaurate, | 8.6^{a} | Croda, Plainsboro, NJ |
| CAS No. 1338-39-2) | | |
| Laureth-3 (2-[2-[2- | 8.0^{f} | Alzo International, |
| (dodecyloxy)ethoxy]ethoxy]- | | Sayreville, NJ |
| ethanol, CAS No. 3055-94-5) | | |
| 1,2-octanediol (CAS No. 1117-86-8) | 7.0^{b} | Parchem, New Rochelle, NY |
| POLYALDO 10-1-S (polyglyceryl-10-monostearate, | 10^{a} | Lonza, South Plainfield, NJ |
| CAS No. 79777-30-3) | | |
| POLYALDO 10-2-P (polyglyceryl-10-dipalmitate) | 11^{a} | Lonza, South Plainfield, NJ |
| SPAN 60 (sorbitan monostearate, | 4.7^{a} | Croda, Plainsboro, NJ |
| CAS No. 1338-41-6) | | |
| NATSURF 265-LQ (ethoxylate of a short | 13.6^{a} | Croda, Plainsboro, NJ |
| chain linear fatty alcohol) | | |
| ISOLAN PDI (diisostearoyl polyglyceryl- | ca. 5^{a} | Evonik, Essen, Germany |
| 3 dimer dilinoleate) | | |
| Polyglyceryl-3-oleate (CAS No. 33940-98-6) | ca. 5^{a} | Evonik, Essen, Germany |
| ISOLAN GI 34 (polyglyceryl-4-isostearate, | ca. 5^{a} | Evonik, Essen, Germany |
| CAS No. 91824-88-3) | | |
| ISOLAN IS (methylglucose isostearate, | ca. 5^{a} | Evonik, Essen, Germany |
| CAS No. 206451-21-0) | | |
| Polyglyceryl-2-sesquioleate | ca. 4^{a} | Evonik, Essen, Germany |
| CITHROL GMIS 40-LQ (glyceryl isosterate, | 3.4^{a} | Croda, Plainsboro, NJ |
| CAS No. 66085-00-5) | | |
| Oleyl alcohol (CAS No. 143-28-2) | 14.5^{a} | Croda, Plainsboro, NJ |
| Dipropylene glycol (CAS No. 25265-71-8) | 9.2^{c} | Sigma Aldrich, St. Louis, MO |
| Diethylene glycol monoethyl ether | 4.2^{d} | Alfa Aesar, Haverhill, MA |
| (CAS No. 111-90-0) | | |
| Lauryl lactate (CAS No. 6283-92-7) | 13.5-14^{a} | Croda, Plainsboro, NJ |
| Oleic acid (CAS No. 112-80-1) | 1.0^{a} | Croda, Plainsboro, NJ |
| Methyl laurate (CAS No. 111-82-0) | 3.7^{a} | Sigma Aldrich, St. Louis, MO |
| SPAN 80 (sorbitan monooleate, CAS No. 1338-43-8) | 4.3^{a} | Croda, Plainsboro, NJ |

^{a}HLB value reported by supplier
^{b}Alany, R. G., International Journal of Pharmaceutics (2000), 196, pages 141-145
^{c}Zhang, W., Minerals (2012), 2, pages 208-227
^{d}Truong, D. H., AAPS PharmSciTech, 17, pages 466-473
^{e}Park, K-M, Food Science and Biotechnology (2018), 27, pages 401-409
^{f}MakingCosmetics, Inc. (Redmond, WA), Specification Sheet for Laureth-3

TABLE 3
Aminosilicone Additives

| Additive | Amine Value | Source |
|---|---|---|
| GP-316 | 54^{g} | Genesee Polymers, |
| | | Burton, MI |
| SILAMINE | 170^{g} | Siltech Corporation, |
| D2 EDA | | Toronto, Canada |
| KF-861 | 50^{h} | Shin-Etsu Silicones, |
| | | Tokyo, Japan |
| KF-8004 | 67^{h} | Shin-Etsu Silicones, |
| | | Tokyo, Japan |
| GP-581 | 110^{g} | Genesee Polymers |
| GP-4 | 90^{g} | Genesee Polymers |
| KF-867S | 59^{h} | Shin-Etsu Silicones |
| GP-344 | 27^{g} | Genesee Polymers |

^{g}amine value reported by supplier
^{h}amine value calculated from functional group molecular weight of the amine groups (g/mol) reported by the supplier

TABLE 4
Cationic Silicone Polyquaternium Additives

| Additive | Description and Source |
|---|---|
| PECOSIL AD-3640 | Silicone Quaternium 8; Phoenix |
| | Chemicals, Sommerville, NJ |
| PECOSIL CA-1240 | Silicone Quaternium 12; |
| | Phoenix Chemicals |
| ZENESTER-Q | Silicone Quaternium 19; |
| | TC-USA Inc., Wilmington, DE |

### Gel Compositions with a Nonionic Surfactant Additive

The components HMDSO (11 g), MQ Resin (1 g), SPOx (1 g), salicylic acid (0.075 g) and a nonionic surfactant (0.4 g) as listed in Table 5 were combined in a vial and mixed with stirring at 60° C. until the SPOx component was completely dissolved in the mixture. An additional portion of SPOx (1.3 g) was added to the vial and the resulting mixture was stirred for 4-6 hours at 60° C.

The vial was cooled to room temperature and then placed on a bench without any agitation of the gel composition for a minimum of 24 hours. Next, the gel composition was inspected by visual examination to determine if the components of the composition were dissolved or uniformly dispersed throughout the gel and noted accordingly.

Then, the gel compositions were individually coated onto the surface of LEXAN polycarbonate test sheets (5.1 cm by 12.7 cm) (a single composition coated per test sheet). About 2-3 mL of a gel was applied to a test sheet using a syringe. A pull-down hand coater was used with a gap set at 50 mil (1.27 millimeter) using a feeler gauge. Coated sheets were air dried overnight and then observed for the presence of particulates. The nonionic surfactant additive used in each sample and the observations made of the gel and the dried film are reported in Table 5.

TABLE 5

| | Nonionic | | Observation | |
|---|---|---|---|---|
| Sample | Surfactant | HLB | Gel | Dried |
| No. | Additive | Value | Composition | Film |
| 1 | Oleic acid | 1.0 | Many visible | Many visible |
| | | | particulates | particulates |
| 2 | CITHROL | 3.4 | Many visible | coating not |
| | GMIS | | particulates | prepared |
| | 40-LQ | | | |
| 3 | Methyl | 3.7 | Many visible | Many visible |
| | laurate | | particulates | particulates |
| 4 | Polyglyceryl-2- | ca. 4 | phase separation | Many visible |
| | sesquioleate | | and visible | particulates |
| | | | particulates | |
| 5 | Diethylene | 4.2 | phase separation | coating not |
| | glycol | | and visible | prepared |
| | monoethyl | | particulates | |
| | ether | | | |
| 6 | SPAN 80 | 4.3 | Many visible | Many visible |
| | | | particulates | particulates |
| 7 | SPAN 60 | 4.7 | Many visible | Many visible |
| | | | particulates | particulates |
| 8 | ISOLAN | ca. 5 | A few visible | A few visible |
| | GI 34 | | particulates | particulates |
| 9 | ISOLAN IS | ca. 5 | A few visible | A few visible |
| | | | particulates | particulates |
| 10 | Polyglyceryl-4- | 6.0 | No visible | No visible |
| | stearate | | particulates | particulates |
| 11 | BRIJ | 6.6 | No visible | No visible |
| | O3-LQ | | particulates | particulates |
| 12 | SENSIVA | 7.0-7.5 | No visible | No visible |
| | SC-10 | | particulates | particulates |
| 13 | Glycerol | 7.0 | No visible | No visible |
| | monolaurate | | particulates | particulates |
| 14 | 1,2- | 7.0 | No visible | No visible |
| | octanediol | | particulates | particulates |
| 15 | SENSIVA | 7.5 | No visible | No visible |
| | SC-50 | | particulates | particulates |
| 16 | Polyglyceryl- | 8.0 | No visible | No visible |
| | 6-distearate | | particulates | particulates |
| 17 | Laureth-3 | 8.0 | No visible | No visible |
| | | | particulates | particulates |
| 18 | SPAN 20 | 8.6 | No visible | No visible |
| | | | particulates | particulates |
| 19 | Brij | 9.1 | A few visible | A few visible |
| | O5-LQ | | particulates | particulates |
| 20 | Dipropylene | 9.2 | Many visible | coating not |
| | glycol | | particulates | prepared |
| 21 | POLYALDO | 10 | Large amount | coating not |
| | 10-1-S | | of solids | prepared |
| 22 | POLYALDO | 11 | Large amount | Many visible |
| | 10-2-P | | of solids | particulates |
| 23 | Lauryl | 13.5-14 | Phase | Many visible |
| | lactate | | separation | particulates |
| 24 | NATSURF | 13.6 | phase | coating not |
| | 265-LQ | | separation | prepared |
| 25 | Oleyl | 14.5 | phase | Many visible |
| | alcohol | | separation | particulates |

### Gel Compositions with an Aminosilicone Additive

The components HMDSO (11 g), MQ Resin (1 g), SPOx (1 g), salicylic acid (0.075 g) and an aminosilicone (0.4 g) as listed in the table below were combined in a vial and mixed with stirring at 60° C. until the SPOx component was completely dissolved in the mixture. An additional portion of SPOx (2 g) was added to the vial and the resulting mixture was stirred for 4-6 hours at 60° C. The vial was cooled to room temperature and then placed on a bench without any agitation of the gel composition for a minimum of 24 hours. Next, the gel composition was inspected and the observations noted.

Gel compositions were individually coated onto the surface of LEXAN polycarbonate test sheets and subsequently inspected and the observations noted. The aminosilicone additive used in each sample and the observations made of the gel and the dried film are reported in Table 6.

TABLE 6

| | Amino- | Amine Value | Observation | |
|---|---|---|---|---|
| Sample | silicone | of Amino- | Gel | Dried |
| No. | Additive | silicone | Composition | Film |
| 26 | Silamine | 170 | No visible | No visible |
| | D2 EDA | | particulates | particulates |
| 27 | GP-581 | 110 | No visible | No visible |
| | | | particulates | particulates |
| 28 | GP-4 | 90 | No visible | No visible |
| | | | particulates | particulates |
| 29 | KF-8004 | 67 | A few visible | A few visible |
| | | | particulates | particulates |
| 30 | KF-867S | 59 | Many visible | Many visible |
| | | | particulates | particulates |
| 31 | GP-316 | 54 | Many visible | Many visible |
| | | | particulates | particulates |
| 32 | KF-861 | 50 | Many visible | Many visible |
| | | | particulates | particulates |
| 33 | GP-344 | 27 | Many visible | (coating |
| | | | particulates | not prepared) |

### Gel Compositions with a Cationic Silicone Polyquaternium Additive

The components HMDSO (11 g), MQ Resin (1 g), SPOx (1 g), salicylic acid (0.075 g) and a polyquaternium (0.4 g) as listed in the table below were combined in a vial and mixed with stirring at 60°° C. until the SPOx component was completely dissolved in the mixture. An additional portion of SPOx (2 g) was added to the vial and the resulting mixture was stirred for 4-6 hours at 60° C. The vial was cooled to room temperature and then placed on a bench without any agitation of the gel composition for a minimum of 24 hours. Next, the gel composition was inspected and the observations noted.

Gel compositions were individually coated onto the surface of LEXAN polycarbonate test sheets and subsequently inspected and the observations noted. The cationic silicone polyquaternium additive used in each sample and the observations made of the gel and the dried film are reported in Table 7.

TABLE 7

| Sample | Polyquaternium | Observation | |
|---|---|---|---|
| No. | Additive | Gel Composition | Dried Film |
| 34 | PECOSIL AD-3640 | No visible | No visible |
| | | particulates | particulates |
| 35 | PECOSIL CA-1240 | No visible | No visible |
| | | particulates | particulates |
| 36 | ZENESTER Q | Very few visible | Very few visible |
| | | particulates | particulates |

### Sample 37: Gel Composition with a Nonionic Surfactant Additive and a Cationic Silicone Polyquaternium Additive

The components HMDSO (9 g), MQ Resin (1 g), SPOx (2.4 g), salicylic acid (0.075 g), SENSIVA-SC10 (0.2 g), and PECOSIL AD-3640 (0.2 g) were combined in a vial and mixed with stirring at 60° C. until the SPOx component was completely dissolved in the mixture. An additional portion of SPOx (2 g) was added to the vial and the resulting mixture was stirred for 4-6 hours at 60° C. The vial was cooled to room temperature and then placed on a bench without any agitation of the gel composition for a minimum of 24 hours. Next, the gel composition was inspected and observations noted.

The gel composition was coated onto the surface of a LEXAN polycarbonate test sheet and subsequently inspected and the observations noted. No visible particulates were observed in the gel composition nor the dried film.

### Sample 38. Gel Composition with a Nonionic Surfactant Additive and a Cationic Silicone Polyquaternium Additive

The same procedure as reported in Sample 37 was followed with the exception that in the composition SENSIVA-SC10 (0.2 g) was replaced with SENSIVA-SC50 (0.2 g). No visible particulates were observed in the gel composition nor the dried film.

### Sample 39. Gel Composition with a Nonionic Surfactant Additive and a Cationic Silicone Polyquaternium Additive

The same procedure as reported in Sample 37 was followed with the exception that in the composition SENSIVA-SC10 (0.2 g) was replaced with SENSIVA-SC50 (0.1 g) and a larger amount of PECOSIL AD-3640 (0.3 g) was used. No visible particulates were observed in the gel composition nor the dried film.

### Sample 40. Gel Composition with Two Nonionic Surfactant Additives

The same procedure as reported in Sample 37 was followed with the exception that in the composition SENSIVA-SC10 (0.2 g) was replaced with BRIJ O5-LQ (0.1 g) and PECOSIL AD-3640 (0.2 g) was replaced with polyglyceryl-4-stearate (0.3 g). No visible particulates were observed in the gel composition nor the dried film.

### Sample 41. Gel Composition with Two Nonionic Surfactant Additives

The same procedure as reported in Sample 40 was followed with the exception that in the composition BRIJ O5-LQ (0.1 g) was replaced with BRIJ O3-LQ (0.1 g). No visible particulates were observed in the gel composition nor the dried film.

### Sample 42. Gel Composition with Two Nonionic Surfactant Additives

The same procedure as reported in Sample 37 was followed with the exception that in the composition SENSIVA-SC10 (0.2 g) was replaced with SPAN 20 (0.1 g) and PECOSIL AD-3640 (0.3 g) was replaced with polyglyceryl-6-stearate (0.3 g). No visible particulates were observed in the gel composition nor the dried film.

### Sample 43. Gel Composition with Two Nonionic Surfactant Additives

The same procedure as reported in Sample 42 was followed with the exception that in the composition SPAN 20 (0.1 g) was replaced with Laureth-3 (0.1 g). No visible particulates were observed in the gel composition nor the dried film.

### Sample 44

Propionibacterium acnes (P. acnes) ATCC 6919 was obtained from ATCC (Manassas, VA). A single colony of P. acnes from a stock agar culture was inoculated into DIFCO anaerobic broth (Becton, Dickinson, Franklin Lakes, NJ) and incubated at 37° C. for 18 hours to provide a 1×10⁹ cfu/mL (colony forming units per milliliter) culture of P. acnes.

Circular discs (1.2 cm diameter) were punched from the coated polycarbonate test sheets prepared in Samples 16, 18, 27, 34, 37-39. For each disc type, three replicates were prepared and tested (n=3). A single disc was placed in a sterile 100×15 mm plastic Petri dish and oriented so that the uncoated surface of the disc contacted the base of the dish. An aliquot (40 microliters) of the P. acnes sample was deposited on the coated surface of each disc using a micropipette. Each disc then covered with a sterile, glass microscope slide coverslip. Control samples were also prepared by depositing a 40 microliter aliquot of the P. acnes sample directly on the base surface of a Petri dish that did not contain an added disc. The added aliquot was subsequently covered with a sterile, glass microscope slide coverslip. Each Petri dish was placed in an incubator at room temperature for 24 hours. After removal from the incubator, the cover slip and disc were separated from each other while still keeping them in the Petri dish. An aliquot (10 mL) of phosphate buffered saline (PBS) (1×, Thermo Fisher Scientific, Waltham, MA) was added to each Petri dish and the Petri dish was shaken for 20 minutes at 100 rpm (revolutions per minute) using a MAXQ Model 8000 orbital shaker (Thermo Fisher Scientific). The resulting PBS solution was serially diluted (10-fold dilutions with PBS) and 3 microliters of each dilution sample was added by pipette to a DIFCO anaerobic agar plate (Becton Dickinson). The individually inoculated agar plates were incubated at 37°° C. for 16 hours. The colonies from each incubated plate were counted by visual examination. The cfu counts of the individual plates (n=3) were averaged and the average count value was used to calculate (based on serial dilutions) the number of colony forming units per milliliter (cfu/mL) that were recovered from each type of inoculated disc. For each sample disc, the logarithmic reduction value (LRV) was calculated according to Equation 1. The results are reported in Table 8. An LRV reported as >6 in Table 8 indicates that no colonies were observed in any of the dilution samples for the example disc.

$$LRV = log 10 [ \frac{concentration of P . acnes recovered from the control sample ( cfu / mL )}{concentration of P . acnes recoved from the Example disc ( cfu / mL )} \qquad Equation 1$$

TABLE 8

| Discs from | Log10 Reduction |
|---|---|
| Sample | Value (LRV) |
| 16 | >6 |
| 18 | >6 |
| 27 | >6 |
| 34 | 1.2 |
| 37 | >6 |
| 38 | >6 |
| 39 | >6 |

Foreseeable modifications and alterations of this invention will be apparent to those skilled in the art without departing from the scope and spirit of this invention. This invention should not be restricted to the embodiments that are set forth in this application for illustrative purposes. To the extent that there is any conflict or discrepancy between this specification as written and the disclosure in any document mentioned or incorporated by reference herein, this specification as written will prevail.

1. A composition for use as a treatment article, the composition comprising:
salicylic acid;
a silicone containing film-forming polymer;
a silicate tackifying resin; and
an additive, wherein the additive comprises (i) a nonionic surfactant having an HLB of 5-9, (ii) an aminosilicone having an amine value greater than 60, (iii) a cationic silicone polyquaternium, or (iv) combinations thereof.

2. The composition according to claim 1, wherein the silicone containing film-forming polymer comprises a linear polydiorganosiloxane.

3. The composition according to claim 1, wherein the silicate tackifying resin is an MQ silicate tackifying resin.

4. The composition according to claim 1, wherein the nonionic surfactant comprises an HLB of 6-8.

5. The composition according to claim 1, wherein the nonionic surfactant comprises at least one of a glyceryl stearate, a polyoxyethylene oleyl ether, glycerol monolaurate, 2-ethylhexyl glycerin ether, 1, 2-alkanediols, and combinations thereof.

6. The composition according to claim 1, wherein the aminosilicone has the structure 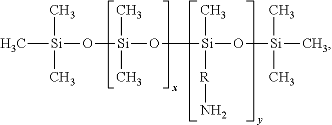
wherein R is an alkyl containing between 1 and 12 carbons, the blocks bearing the subscripts x and y may be randomly mixed, the total value of x is from 10 to 5,000, and the total value of y is from 2 to 20.

7. The composition according to claim 1, wherein the aminosilicone has the structure 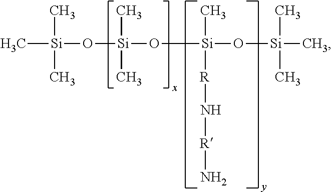
wherein the blocks bearing the subscripts x and y may be randomly mixed, the total value of x is from 5 to 5,000, the total value of y is from 1 to 20, R and R′ may be the same or different, and R and R′ are each saturated, linear or branched alkyl groups of 1 to 12 carbon atoms.

8. The composition according to claim 1, wherein the cationic silicone polyquaternium comprises at least one of silicone quaternium-12, silicone quaternium-8, silicone quaternium-19, silicone quaternium-22, and mixtures thereof.

9. The composition according to claim 1, wherein the composition comprises at least 0.5 and at most 30wt % of the salicylic acid.

10. The composition according to claim 1, wherein the composition comprises at least 0.1 and at most 10 at % of the salicylic acid.

11. The composition according to claim 1, wherein the composition further comprises a volatile solvent, optionally wherein the volatile solvent is isooctane or hexamethyldisiloxane.

12. The composition according to claim 1, wherein the composition further comprises a filler.

13. The composition according to claim 1, wherein the composition further comprises a coagulant.

14. The composition according to claim 1, wherein the composition comprises at least 2 wt % of the additive.

15. The composition according to claim 1, wherein the composition comprises:
0.1 to 10 wt % salicylic acid;
5 to 30 wt % silicone containing film-forming polymer;
1 to 35 wt % silicate tackifying resin;
0.1 to 5 wt % additive; and
50 to 80 wt % volatile solvent,
each based on the total weight of the composition.

16. The composition according to claim 1, wherein the film composition comprises comprising
0.1 to 20 wt % salicylic acid;
30 to 90 wt. % silicone containing film-forming polymer;
5 to 40 wt. % silicate tackifying resin; and
0.1 to 25 wt % additive,
each based on the total weight of the composition.

17. A composition for use as a skin treatment, wherein the composition is according to claim 1.

18. A method of making a gel composition, the method comprising:
combining a first part and a second part to make the gel composition, wherein the gel composition comprises
salicylic acid;
a silicone containing film-forming polymer;
a silicate tackifying resin;
a volatile solvent; and
an additive, wherein the additive comprises an additive, wherein the additive comprises (i) a nonionic surfactant having an HLB of 5-9, (ii) an aminosilicone having an amine value greater than 60, (iii) a cationic silicone polyquaternium, or (iv) combinations thereof;
wherein the first part comprises the salicylic acid in a first portion of the silicone containing, film-forming polymer; and the second part comprising a second portion of the silicone containing, film-forming polymer.

19. A method comprising:
exposing tissue containing acne bacteria to a composition, the composition comprising:
salicylic acid;
a silicone containing film-forming polymer;
a silicate tackifying resin; and
an additive, wherein the additive is (i) a nonionic surfactant having an HLB of 5-9, (ii) a aminosilicone having an amine ratio greater than 0.05, (iii) a cationic silicone polyquaternium, or (iv) combinations thereof.
